(19) United States
(12) Patent Application Publication
(10) Pub. No.: US 2011/0218524 A1
(43) Pub. Date: Sep. 8, 2011

(54) METHOD AND APPARATUS FOR LASER-BASED SURGERY AND TREATMENT

(75) Inventor: Giorgio CATTANEO, Karlsruhe (DE)

(73) Assignee: ACANDIS GMBH & CO. KG, Pfinztal (DE)

(21) Appl. No.: 12/717,283

(22) Filed: Mar. 4, 2010

(51) Int. Cl. A61B 18/22

(52) U.S. Cl. 606/12; 606/15

(57) ABSTRACT

The present invention relates to a method and apparatus for laser-based surgery and treatment, in particular of the human body. Inter alia, the present disclosure teaches an apparatus having a femtosecond laser that emits at least one pulse of laser energy having a pulse duration of less than 100 femtoseconds and a fiber optical channel for conducting said emitted pulse of laser energy to a vicinity of a human body to irradiate a localized area of said human body to effect at least one of microsurgery, neurosurgery, treatment of cardiovascular disease, treatment of the skin, tissue removal for biopsy, cutting of a mucous membrane, disintegration or vaporization of a gallstone or a kidney stone, and orthopedic surgery.

### BACKGROUND OF THE INVENTION

1. Field of the Invention

The present invention relates to a method and apparatus for laser-based surgery and treatment, in particular of the human body. More specifically, it relates to the use of a femtosecond laser in such a method/apparatus.

2. Description of the Related Art

Many techniques and devices are known for performing surgery or treatment on the human body. All of these known techniques and devices are plagued by the fact that they inherently affect an unduly large region of tissue relative to many small structures as found e.g. in the human brain or the human cardio-vascular system. In other words, conventional techniques and devices do not provide the precision required by doctors and surgeons to avoid “collateral damage” to healthy tissue and structures in the immediate vicinity of the area of treatment/surgery.

It is an object of the present disclosure to provide both a method and apparatus for surgery/treatment that overcomes the aforementioned deficiencies of the prior art. It is likewise an object of the present disclosure to teach previously unforeseen techniques for treating the human body.

### BRIEF SUMMARY OF THE INVENTION

In accordance with a first aspect, the present disclosure provides an apparatus having a femtosecond laser that emits at least one pulse of laser energy having a pulse duration of less than 100 femtoseconds and a fiber optical channel for conducting said emitted pulse of laser energy to a vicinity of a human body to irradiate a localized area of said human body to effect at least one of:
microsurgery, in particular separation of tumor tissue from said human body, in particular from a brain of said human body,
neurosurgery, e.g. capping of a nerve, separation of at least one of an epineurium and a perineurium from a nerve, capping of a cardiac nerve associated with an arrhythmia, capping of a renal nerve associated with blood pressure regulation,
treatment of cardiovascular disease, e.g. removal of calcification (e.g. from a heart valve), tissue modification of vulnerable plaque, stimulation of vessel regions (e.g. stimulation of a baroreceptor), sectioning of vessels in preparation for anastomosis or bypass,
treatment of the skin,
tissue removal for biopsy,
treatment of birthmarks or moles,
cutting of a mucous membrane, e.g. in paranasal sinuses or a nasal cavity,
disintegration or vaporization of a gallstone or a kidney stone, and
orthopedic surgery.

In accordance with a second aspect, the present disclosure provides a corresponding method for effecting any of the aforementioned treatments.

In accordance with a third aspect, the present disclosure teaches use of a femtosecond laser for manufacturing an apparatus for effecting any of the aforementioned treatments.

### BRIEF DESCRIPTION OF THE DRAWINGS

The novel features of the invention, as well as the invention itself, both as to its structure and its operation will be best understood from the accompanying figures, taken in conjunction with the accompanying description. The Figures show:

FIG. 1 an apparatus in accordance with the present disclosure

### DETAILED DESCRIPTION OF THE INVENTION

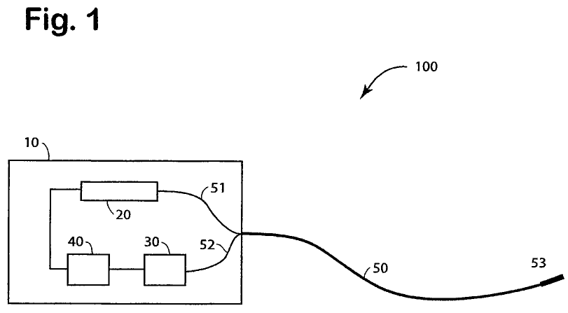

FIG. 1 shows an apparatus 100 having an optional housing 10. The apparatus 100 comprises a femtosecond laser 20 configured and adapted to output at least one pulse of laser energy. The pulse of laser energy has a duration on the order of femtoseconds, e.g. a duration of less than 100 fs, 50 fs, 10 fs, 5 fs or even 1 fs. The pulse of laser energy is followed by a period in which femtosecond laser 20 does not emit laser energy. This period is at least twice as long as the duration of the preceding pulse of laser energy, yet is typically on the order of several microseconds or larger.

In accordance with the embodiment shown in FIG. 1, the pulse of laser energy emitted by femtosecond laser 20 is coupled into an optical channel 51 of a fiber-optic cable 50. As known in the art of fiber-optic cables, optical channel 51 conducts the laser energy from one end of optical channel 51 to the other end of optical channel 51 with negligible loss. Fiber-optic cable 50 has a length suitable for transmitting the pulse of laser energy from femtosecond laser 20 to the vicinity of or deeply into the cardiovascular system of a patient requiring treatment, e.g. a length of 2 to 6 meters. To allow transmission of the pulse of laser energy deeply into the cardiovascular system, fiber-optic cable 50 can be configured with an extremely small outer diameter, e.g. on the order of 100 to 500 μm. Similarly, fiber-optic cable 50 can be configured to be flexible along its entire length or along a portion of its length, e.g. along at least a portion that is intended to be inserted into the cardiovascular system. In the present context, “flexible” is to be understood as having a small bending radius, e.g. a bending radius on the order of 3-20 mm. The bending radius indicates how sharply the fiber-optic cable 50 can be bent without damaging the fiber-optic cable 50. Preferably, the bending solely incurs elastic deformation of the fiber-optic cable 50, i.e. incurs no plastic or other irreversible deformation of the fiber-optic cable 50.

To enhance steerability of fiber-optic cable 50 to the area requiring treatment (e.g. through various passages of the patient's cardiovascular system) and to enhance the accuracy with which the laser energy transmitted through optical channel 51 from femtosecond laser 20 can be aimed at a particular region of the patient's body, fiber-optic cable 50 can be provided with a stiff, i.e. substantially inflexible, distal end portion 52. Choosing an appropriate length for stiff distal end portion 53 involves balancing the desire for flexibility and the desire for steerability/aiming accuracy. For an embodiment of a fiber-optic cable 50 for insertion into the cardiovascular system, a length in the range of 3 to 10 mm for stiff distal end portion 53 has been determined to be appropriate. Similarly, the flexibility of the fiber-optic cable 50 can vary anywhere along the length of the fiber-optic cable 50, e.g. can vary continuously.

Aiming of the laser energy transmitted through optical channel 51 from femtosecond laser 20 at a particular region of the patient's body can likewise be accomplished by providing a movable, e.g. a steerable lens at the distal end of optical channel 51. A steerable lens is of utility e.g. for treating plaque or stenoses along the wall of a vessel that thus surround the tip of the fiber-optic cable 50. It such cases, it is desirable to direct the laser energy generally in a radially outward direction relative to the axis of the fiber-optic cable 50 rather than in an axial direction.

For the sake of obtaining feedback with regard to the laser treatment, fiber-optic cable 50 can comprise a feedback optical channel 52 that is configured to capture laser light that has been emitted from the distal end of optical channel 51 and has then been reflected back to the distal end of feedback optical channel 52 by the tissues/structures under treatment or by surrounding tissues/structures. The laser light captured at the distal end of optical channel 52 is then transmitted through fiber-optic cable 50 to an optical processing apparatus 30 where it is subjected to processing, e.g. spectrometric analysis. Such processing/analysis of the light obtained from the area of treatment or its vicinity via feedback optical channel 52 can yield information regarding the type of tissue/structure being irradiated by the laser energy transmitted through optical channel 51, which can be indicative of the state of progress of the treatment. For example, light reflected from a gallstone will exhibit a different spectral characteristic than light reflected from surrounding tissue. Accordingly, a sudden or sharp decrease in the amount of light received via feedback optical channel 52 having a spectral characteristic indicative of reflection off a gallstone can be indicative of incorrect aiming of the irradiated laser energy or of disintegration of gallstone material previously located in the beam path of the laser energy irradiated from the distal end of optical channel 51. In either case, a (similarly marked) decrease in the amount of laser energy irradiated per unit of time could be appropriate to prevent unintentional damage to healthy tissue in the beam path.

As shown in FIG. 1, apparatus 100 may comprise a control apparatus 40 that is communicatively coupled to optical processing apparatus 30 and femtosecond laser 20. Control apparatus 40 can be configured to automatically control an output of femtosecond laser 20 based on an input obtained from optical processing apparatus 30 that is indicative of a result obtained by processing the light captured by feedback optical channel 52 as described above. Control apparatus 40 can control the output of femtosecond laser 20 e.g. as regards the pulse duration of laser energy pulses emitted from femtosecond laser 20, the power of each laser energy pulse emitted from femtosecond laser 20, the number of pulses per unit of time, the total number of pulses, etc.

Naturally, apparatus 100 can be configured and adapted to display data indicative of a result obtained by processing the light captured by feedback optical channel 52 as described above to a user. Such a display of data to a user can be in addition to or in lieu of the aforementioned communication of data from optical processing apparatus 30 to control apparatus 40. Similarly, apparatus 100 can be configured and adapted to receive input from a user regarding control of the femtosecond laser, e.g. as regards the pulse duration of laser energy pulses emitted from femtosecond laser 20, the power of each laser energy pulse emitted from femtosecond laser 20, the number of pulses per unit of time, the total number of pulses, etc. Such user control of the femtosecond laser 20 can be in addition to or in lieu of the aforementioned control of the femtosecond laser 20 by control apparatus 40.

Femtosecond laser 20 can be controlled so as to emit at least one pulse of laser energy having a power in the range of 0.5 to 5 μJ per pulse. Due to the limited amount of energy emitted per pulse, a volume of less than 1 cubic millimeter of tissue (or other bodily structure) will be affected per pulse. Indeed, femtosecond laser 20 can be controlled such that the volume of affected tissue, depending on the type of tissue/structure being treated, lies in the range of 0.001 cubic millimeters to 0.05 cubic millimeters per pulse. The volume of affected tissue per pulse can even be as low as 1 cubic μm. Accordingly, the volume of affected tissue per pulse can likewise lie in the range of 1 cubic μm to 5 cubic μm, in the range of 5 cubic μm to 10 cubic μm or in the range of 10 cubic μm to 100 μm. As a result, apparatus 100 opens the door to a new dimension of microsurgery including previously unforeseen types of surgery requiring extreme precision.

In the following, specific aspects of the general disclosure supra will be discussed.

As stated above, conventional laser systems for medical applications are disadvantageous in that they generate undesired heat in tissue and structures in the vicinity of the region under treatment. Accordingly, conventional laser systems are unsuitable for microsurgery.

The apparatuses and techniques disclosed herein allow the destruction of tissue with high precision and in a spatially confined region. This allows cutting in a region without damage to adjacent tissue. By adjusting the penetration depth of the laser energy, e.g. by adjusting the focus of the irradiating laser beam, deep, yet exacting cuts can be made.

The dimensions as well as the mechanical characteristics (such as flexibility) of the cable that transmits the laser pulses are decisive in achieving high precision treatment of regions of the body that are difficult to reach.

The present disclosure teaches a transmission of laser pulses, in particular having a duration of the order of femtoseconds, through a very thin cable. The cable exhibits an outer diameter that is smaller than 500 micrometers, e.g. less than 300 μm, 250 μm, 200 μm, 150 μm or 100 μm. The cable can be a cable with a hollow core, a mode field diameter fiber or a photonic-crystal fiber. The thin cable can have the length of a catheter, e.g. a length of up to 2 meters, for example for endovascular treatment. In some cases, e.g. for some types of microsurgery, the cable need only be about 0.1 meters in length. The employment of a cable with such small dimensions is only possible in conjunction with a laser that emits pulses having a duration on the order of femtoseconds by limiting the power per pulse to a very small value, e.g. a power of less than 5, 2, 1 or 0.5 μJ per pulse.

The disclosed cable can be embodied such that at least a portion of the cable that transmits the laser pulse is very flexible. This allows access to regions reachable through tortuous paths. It can also be embodied such that only the (distal) tip of the cable is flexible. This allows regions encountered before the area of treatment to be circumvented as necessary, for example. Similarly, the flexible region can be proximal to the end section and the distal section can exhibit high strength or be very stiff. This allows bodily tissue to circumvented with the entire cable, while simultaneously ensuring high stability at the tip. High stability at the tip ensures that the laser can be accurately aimed during treatment. The flexible catheter region can also be steerable. The laser can be navigated by endoscopic methods.

The laser cable, i.e. the cable that conducts the laser light to an area of treatment, can comprise a movable, steerable lens. Precise ablation without damage to neighboring regions can be achieved by moving the lens while maintaining the cable in a fixed position.

Feedback, e.g. spectrometric feedback, can be provided for recognition of the characteristics of the affected tissue, and the system can comprise a control apparatus e.g. for stopping the emission of laser pulses depending on the feedback or information derived therefrom. This makes it possible to treat tissue having known characteristics while neighboring tissue of a different type remains unaffected by the laser. For example, hard tissue/structures such as bone cells can be removed without the danger of damaging neighboring soft tissue such as vessels and nerves.

The cable/catheter can be provided with at least one flexible region. The flexible region can have a radius of curvature in the range of 3 to 20 mm, e.g. a radius of curvature of less than 20, 15, 10, 8, 5 or 3 mm.

By using a femtosecond laser, i.e. a laser that emits pulses of laser light having a duration on the order of femtoseconds, it is possible to make cuts without adversely affecting neighboring tissue since the generation of heat is spatially localized. Accordingly, it is possible to remove tissue with high precision in both a lateral and an axial (depth) direction. This precision in both a lateral and axial direction allows deep, yet very precise incisions to be carried out.

A summary of diseases and conditions that can be treated by such an apparatus/by such techniques based on a femtosecond laser in conjunction with an extremely thin (and optionally flexible) cable follows.

Neurosurgery/minimal invasive surgery:

The apparatuses and techniques described above can be used for tumor removal, in particular in the region of the brain. Many tumors cannot be removed because injury to the proximal tissue would be unacceptable. By using a thin and flexible cable, areas can be reached for treatment that are otherwise not easily or feasibly accessible. By using a femtosecond laser, it is possible to excise the tumor or parts thereof with extreme precision. Tumors commonly grow into healthy tissue, whence it is often necessary to excise some of the healthy tissue proximal to the tumor to ensure complete removal of the tumorous tissue. By using a femtosecond laser, it is possible to reduce the amount of healthy tissue removed to a minimum.

Naturally, the minimally invasive laser treatment apparatuses and techniques described above can be used for tumor removal from other regions of the body and other types of bodily tissue. Moreover, these apparatuses and techniques can provide novel forms of laser treatment. For example, instead of cutting or excising a tumor, the femtosecond laser can be used to biologically alter the cancerous cells without substantial destruction or removal of tissue. Specifically, the amount of laser energy irradiated onto the area of treatment can be dosed, e.g. as described above, such that the outer structure of the cells remains intact, yet their inner structure is biologically modified.

The apparatuses and techniques described above can be used for treatment of the nervous system, including treatment of nerves and separation of nerves from one another. The separation of nerves is of importance, for example, in the field of pain therapy, e.g. in the treatment of worn-out joints and neurological diseases. Since nerves are often grouped in bundles, conventional surgical techniques almost always result in a cutting of several nerves. It is thus desirable to carefully and exactingly separate the individual nerves that run parallel to one another. This can be achieved by the apparatuses and techniques taught herein. Moreover, the laser is capable of precisely capping one or more individual nerves. The small dimensions of the fiber-optic cable play a decisive role in this respect since sections of other important nerves may run very close to the nerve or nerves to be capped.

The teachings of the present disclosure are similarly applicable to an ablation of the outer membranes of a nerve, e.g. the epineurium or the perineurium, for the sake of treating the nerves encased therein.

Cardiovascular and endovascular treatment:

Clogged, e.g. due to calcification, heart valves in be treated via the precision laser techniques and apparatuses taught herein. By applying the focus of the laser to a very small region (in this case mainly axially, i.e. primarily deeply into the tissue with minimal lateral expanse), the clogging/calcified region can be removed with negligible damage to neighboring healthy tissue. The flexibility and the very small diameter of the cable are advantageous in this respect and since that allows the system to be introduced into the area of treatment endovascularly, e.g. intravenously, from a peripheral vessels, for example from a femoral vein or a fermoral artery (e.g. for the treatment of heart valves in the left ventricle). Employment of such a technique avoids the necessity of surgical treatment of the heart valve. Accordingly, the aorta need not be opened, which avoids the need for connecting the patient to a heart-lung machine and the substantial risks associated therewith.

Clogged blood vessels that limit blood flow, i.e. blood vessels with stenosis, can be treated intravenously by means of a thin microcatheter as described hereinabove. Similarly, blood vessels soft plaque (also known as vulnerable plaque) can be safely treated by transmitting the laser light inside a thin, flexible catheter to the area of treatment. In contrast, mechanical surgery of soft plaque is undesirably dangerous since pieces of plaque can be uncontrollably detached, i.e. can be released into the bloodstream, and can thus lead to obstruction in a remote vessel of the cardiovascular system with a possibly crippling or lethal effect. As discussed above, employment of a femtosecond laser allows ablation of the areas of plaque without damaging the neighboring tissue. Another particular advantage of this technique is that soft plaque and calcified tissue are effectively so finely vaporized by the laser that the vaporized tissue can be absorbed from the bloodstream without problems. The teachings of the present disclosure thus provide considerable advantages over mechanical surgery of soft plaque or intervention through mechanical systems such as stents.

As discussed supra, the techniques and apparatuses of the present disclosure can be used for capping, i.e. severing, nerves, e.g. in the heart for treatment of arrhythmia, without damaging the neighboring tissue. The laser pulse can be introduced into the body endovascularly or an incision can be made in the inner wall of a cardiac chamber. The incision can also be made from outside the body, e.g. using endoscopic techniques. As compared to high-frequency ablation or cryoablation, the teachings of the present disclosure are advantageous inter alia on account of their spatial accuracy and the small dimensions of the instruments involved.

A further, similar application is the capping of nerves in the renal arteries, which allows the blood pressure to be influenced, e.g. for the sake of lowering a patient's blood pressure.

The teachings of the present disclosure can also be used for vaporizing/disintegrating thrombi with high precision.

The teachings of the present disclosure can be used for stimulating specific regions in the cardiovascular system. For example, baroreceptors in the cardiovascular system that are responsible for triggering contraction of vascular muscles and consequently for regulating the flow of blood and for regulating blood pressure can be stimulated by means of the laser.

The teachings of the present disclosure can also be used for surgically cutting vessels in preparation for bypasses or anastomoses.

Treatment of the skin:

Skin diseases and irregularities (e.g. as a result of acne, calluses or wrinkles) can be treated by means of a femtosecond laser as taught hereinabove.

The teachings of the present disclosure can also be used for carrying out a biopsy, e.g. with respect to a birthmark, mole or other skin irregularity suspected of being carcinogenic or cancerous, without damaging the neighboring tissue and without stimulating what may be malignant tissue. This approach also significantly reduces bleeding.

The teachings of the present disclosure are also of particular utility for making precise incisions in mucous membranes, e.g. as found in the nose and the paranasal sinuses.

Orthopedic surgery:

In the case of arthrosis and many forms of arthritis, there is a wearing of the joints, for example in the knees, hips, wrists or thumb region. The cartilage that covers the bones in the region of the joint protects each bone from wear during the relative motion with respect to the other bone(s) in the joint. When the cartilage is worn, some portions of the bones in the joint contact one another, which can lead to unphysiological growth in the region of contact. Bone protuberances and other irregularities can result that lead to considerable restrictions of mobility and a considerable pain. Using a femtosecond laser as taught herein, it is possible to ablate such protuberances and irregularities endoscopically in a minimally invasive fashion. In the case of implantation of an artificial joint, it is typically desirable to smooth or otherwise fashion the joint region of the respective bones in a precise manner using ablation. This allows the artificial joint to be correctly positioned and properly fastened. The teachings of the present disclosure can be used for this purpose.

The teachings of the present disclosure are likewise of utility in the treatment of so-called “impingement syndrome.” The term “impingement syndrome” is used to designate impaired joint mobility, specifically arising from degeneration or pinching of capsule or tendinous material. This can arise from a thickening of a tendon (e.g. in the shoulder region) or from anomalies in bone structures (for example as a result of an accident, e.g. to the hip or shoulder) that impede normal joint mobility. The pulses of laser energy can be used to excise or pulverize protuberences or overgrowth.

The teachings of the present disclosure can be similarly used for treatment of the spinal column, e.g. for ablation of tissue components (e.g. from the nucleus and/or annulus) of the discs or for ablation of bone constituents of the spinal column.

Treatment of the kidneys or gallbladder:

The teachings of the present disclosure can also be employed for disintegration of kidney stones, gallstones and the like. Feedback of the laser can be carried out in such a fashion that only the hard substances are disintegrated while the adjacent soft organ tissue is kept intact.

Biopsies:

The use of a femtosecond laser as taught herein provides significant advantages as regards obtaining small tissue samples from various organs and various regions of the body, in particular those that are difficult to access. In the case of tumor cells, for example, tissue is not spread. The sample taking is also less traumatic on account of the smaller and more precise cut. Indeed, it becomes possible to obtain tissue samples from regions of the human body that are not feasibly accessible by previously known techniques.

While various embodiments of the present invention have been disclosed and described in detail herein, it will be apparent to those skilled in the art that various changes may be made to the configuration, operation and form of the invention without departing from the spirit and scope thereof.

1. A method of cerebral microsurgery, comprising the steps of:
providing a treatment apparatus comprising a femtosecond laser that supplies at least one pulse of laser energy having a pulse duration of less than 100 femtoseconds;
conducting said pulse of laser energy by means of a fiber optical cable to a vicinity of a brain in a human body;
irradiating said pulse of laser energy onto said brain to effect cutting of a localized area of said brain. ,

2. The method of claim 1, wherein said localized area has a volume of less than 1 cubic millimeter per pulse.

3. The method of claim 1, wherein said pulse of laser energy has a power of less than 5 μJ.

4. A method of micro-neurosurgery, comprising the steps of:
providing a treatment apparatus comprising a femtosecond laser that supplies at least one pulse of laser energy having a pulse duration of less than 100 femtoseconds;
conducting said pulse of laser energy by means of a fiber optical cable to a vicinity of a nerve in a human body;
irradiating said pulse of laser energy onto said nerve to effect cutting of a localized area of said nerve.

5. The method of claim 4, wherein said localized area has a volume of less than 1 cubic millimeter per pulse.

6. The method of claim 4, wherein said pulse of laser energy has a power of less than 5 μJ

7. The method of claim 4, wherein said nerve is associated with an arrhythmia of a heart of said human body.

8. The method of claim 7, wherein said conducting step comprises introducing said fiber optical cable into said heart via a lumen of a cardiovascular system of said human body

9. The method of claim 4, wherein said nerve is a renal arterial nerve that influences blood pressure in said human body.

10. The method of claim 4, comprising the step of irradiating at least one pulse of laser energy from said femtosecond laser having a pulse duration of less than 100 femtoseconds onto at least one of an epineurium and a perineurium of said nerve to effect a removal of said at least one of an epineurium and a perineurium from said nerve.

11. A method of cardiovascular microsurgery, comprising the steps of:
providing a treatment apparatus comprising a femtosecond laser that supplies at least one pulse of laser energy having a pulse duration of less than 100 femtoseconds;
conducting said pulse of laser energy by means of a fiber optical cable into a cardiovascular system of a human body;
irradiating said pulse of laser energy onto diseased tissue of said cardiovascular system to effect a disintegration or a vaporization of a localized area of said diseased tissue.

12. The method of claim 11, wherein said localized area has a volume of less than 1 cubic millimeter per pulse.

13. The method of claim 11, wherein said pulse of laser energy has a power of less than 5 μJ.

14. The method of claim 11, wherein said localized area is on a heart valve of said human body.

15. The method of claim 11, wherein said diseased tissue is vulnerable plaque.

16. A method of cardiovascular treatment, comprising the steps of:
providing a treatment apparatus comprising a femtosecond laser that supplies at least one pulse of laser energy having a pulse duration of less than 100 femtoseconds;
conducting said pulse of laser energy by means of a fiber optical cable into a cardiovascular system of a human body;
irradiating said pulse of laser energy onto a baroreceptor of said cardiovascular system to effect a stimulation of said baroreceptor.

17. A method of cardiovascular treatment, comprising the steps of:
providing a treatment apparatus comprising a femtosecond laser that supplies pulses of laser energy, each of said pulses having a pulse duration of less than 100 femtoseconds; conducting said pulses of laser energy by means of a fiber optical cable to a vicinity of a cardiovascular vessel of a human body;
irradiating said pulses of laser energy onto said cardiovascular vessel to effect a sectioning of said cardiovascular vessel.

18. The method of claim 17, comprising the step of:
suturing said sectioned cardiovascular vessel to another cardiovascular vessel of said human body to form an anastomosis or bypass.

19. A method of laser surgery, comprising the steps of:
providing a treatment apparatus comprising a femtosecond laser that supplies at least one pulse of laser energy having a pulse duration of less than 100 femtoseconds;
conducting said pulse of laser energy by means of a fiber optical cable to a vicinity of a mucous membrane in paranasal sinuses or a nasal cavity of a human body;
irradiating said pulse of laser energy onto said mucous membrane to effect cutting of a localized area of said mucous membrane.

20. The method of claim 19, wherein said localized area has a volume of less than 1 cubic millimeter per pulse.

21. The method of claim 19, wherein said pulse of laser energy has a power of less than 5 μJ.

22. A method of laser surgery, comprising the steps of:
providing a treatment apparatus comprising a femtosecond laser that supplies at least one pulse of laser energy having a pulse duration of less than 100 femtoseconds;
conducting said pulse of laser energy by means of a fiber optical cable to a vicinity of an area of irregular tissue on a skin of a human body;
irradiating said pulse of laser energy onto said area of irregular tissue to effect disintegration or vaporization of a localized area of said area of irregular tissue.

23. The method of claim 22, wherein said localized area has a volume of less than 1 cubic millimeter per pulse.

24. The method of claim 22, wherein said pulse of laser energy has a power of less than 5 μJ.

25. The method of claim 22, wherein said area of irregular tissue comprises at least one of diseased tissue, calloused tissue, wrinkled tissue, scarred tissue, pigmented tissue or cancerous tissue.

26. A method of laser-based treatment, comprising the steps of:
providing a treatment apparatus comprising a femtosecond laser that supplies pulses of laser energy having a pulse duration of less than 100 femtoseconds;
conducting said pulses of laser energy by means of a fiber optical cable to a vicinity of a gallstone or kidney stone in a human body;
irradiating said pulses of laser energy onto said gallstone or kidney stone to effect at least partial disintegration or vaporization of said gallstone or kidney stone.

27. The method of claim 26, comprising the steps of:
capturing a portion of said irradiated pulses of laser energy;
subjecting said captured portion of said irradiated pulses of laser energy to spectrometric analysis; and
ceasing said irradiation of said pulses of laser energy if said spectrometric analysis yields a result indicative of irradiation of tissue of said human body with said pulses.

28. A method of orthopedic laser surgery, comprising the steps of:
providing a treatment apparatus comprising a femtosecond laser that supplies pulses of laser energy having a pulse duration of less than 100 femtoseconds;
conducting said pulses of laser energy by means of a fiber optical cable to a vicinity of a joint in a human body;
smoothing a bone surface of said joint by irradiating said pulses of laser energy onto said bone surface to effect at least partial disintegration or vaporization of irregularities on said bone surface.

29. A method of tumor cell devitalization, comprising the steps of:
providing a treatment apparatus comprising a femtosecond laser that supplies at least one pulse of laser energy having a pulse duration of less than 100 femtoseconds;
conducting said pulse of laser energy by means of a fiber optical cable to a vicinity of a tumor cell in a human body;
irradiating said pulse of laser energy onto said tumor cell, wherein said pulse of laser energy is supplied by said femtosecond laser with an energy such that said irradiated pulse has an energy low enough to avoid structurally damaging a cell membrane of said tumor cell and high enough to damage at least one subcellular component of said tumor cell within said cell membrane.

30. The method of claim 29, wherein said at least one subcellular component comprises a nucleus of said tumor cell.
